(19) United States
(12) Patent Application Publication
(10) Pub. No.: US 2023/0407413 A1
(43) Pub. Date: Dec. 21, 2023

(54) PCR-Based Diagnostics With Empiric Antibiotic Selection

(71) Applicant: Brian Joseph Hajjar, Palisades Park, NJ (US)

(72) Inventor: Brian Joseph Hajjar, Palisades Park, NJ (US)

(21) Appl. No.: 18/445,247

(22) Filed: Jun. 16, 2023

Related U.S. Application Data
(60) Provisional application No. 63/473,733, filed on Jun. 18, 2022.

(51) Int. Cl. C12Q 1/689; C12Q 1/6844

(52) U.S. Cl. CPC C12Q 1/689; C12Q 1/6844; C12Q 2600/106

(57) ABSTRACT

The present invention allows for early diagnosis and treatment of an infection using a molecular amplification method with API integration to a database of recommended antimicrobial agents. The method which may be a PCR-based technique determines a microorganism in a biological sample and provides a concurrent determination of empiric antibiotic therapy.

### CROSS REFERENCE TO RELATED APPLICATIONS

This application claims the benefit of priority to U.S. Provisional Application Ser. No. 63/473,733, filed Jun. 18, 2022, the contents of which are incorporated by reference herein in their entirety.

### TECHNICAL FIELD

The present invention is directed to molecular amplification diagnostics for infectious diseases and empiric antimicrobial therapy.

### BACKGROUND

The detection of specific sequences of nucleic acid associated with a particular study of microorganisms, viruses, and/or strains thereof generally requires amplification. Thus, firstly the part of interest of the long nucleic acid molecule is identified, then it is replicated (amplified) by a technique known as polymerase chain reaction (PCR). PCR is a biochemical process capable of amplifying a single DNA molecule into millions of copies in a short time. Use of PCR for specific or broad-spectrum pathogen detection has great clinical application. PCR has been championed by infectious disease experts for identifying organisms that cannot be grown in vitro, or in instances where existing culture techniques are insensitive and/or need prolonged incubation times. In contract with traditional culture-based methods of microbial identification, with reporting of results in hours rather than days, the use of PCR to detect various pathogens saves time.

Despite the proven efficacy and rapid results of the PCR assays for respiratory, genital and urine samples, PCR-based infectious disease diagnostics have not yet resulted in its application to a range of outpatient clinical settings. Conventional methods for pathogen detection have not been supplanted by PCR-based assays because the latter cannot be elaborated to further characterize detected pathogens. To do so in a single reaction, simultaneous amplification of several target genes is needed. Repeating amplifications with different primer pairs, so-called multiplexing, is notoriously difficult since often one or more of the target sequences do not amplify.

Currently, there is still a requirement to grow or plate the organism to determine antimicrobial sensitivity. All of this requires additional time and testing becomes a very lengthy process, leaving the infection unchecked in the patient, leading to increased pathogen load and sometimes the need for hospitalization. The technique of plating has several disadvantages. It requires dedicated equipment and extensive sample preparation, which increases costs and delays the production of results. Consequently, antibiotic therapy is typically begun on an empiric basis, since the causative organism is not yet identified in an appreciable proportion of patients.

Critical and timely intervention for infectious diseases relies on rapid and accurate detection of the pathogen. Informed decision-making for treatment related to such rapid and accurate detection is lacking for PCR. Unfortunately, despite the recognition that outcomes from infectious illnesses are directly associated with time to pathogen identification, and the fact the PCR can provide that rapid pathogen identification, conventional laboratories remain encumbered by traditional, slow multistep culture-based assays, which preclude application of diagnostic test results in the acute, critical-care and community-based settings. Other limitations of the conventional laboratory include extremely prolonged assay times for fastidious pathogens (up to several weeks), requirements for additional testing, and wait times for characterizing detected pathogens (i.e., discernment of species, strain, virulence factors, and antimicrobial resistance).

Traditional methods for identification and antimicrobial susceptibility testing of organisms from clinical specimens typically require overnight subculturing to isolate individual species prior to biochemical assay-based identification, followed by growing isolated organisms in the presence of various antimicrobials to determine susceptibilities. Procedures to obtain these antibiotic susceptibility data are often time-consuming (48-72 hours), cumbersome, and require highly skilled personnel and expensive automatic equipment. PCR identification methods can provide organism identification in a few hours directly from clinical specimens as well as resistance marker detection, but these methods do not provide the antimicrobial susceptibility information required by clinicians to inform treatment decisions. This leaves the clinician to prescribe inadequate or overly-broad spectrum empiric therapy while waiting two to four days for conventional antibiotic susceptibility test results before adjusting therapy.

While available in a few hours, PCR results only provide a partial answer. Processes known in the art are insufficient to efficiently and accurately identify appropriate antimicrobial therapy.

### SUMMARY

The present invention provides devices and methods for determining the presence or absence of pathogens in a biological sample using one or more molecular amplification tests and for the concurrent determination of the empiric oral antimicrobial regimen for pathogens present.

An object of the present invention is to provide a system which can perform PCR analysis in biological samples for microorganisms and match up microorganisms with empiric antibiotic data thereby allowing effective antibiotics to be selected within a short time period.

According to the present invention, a method for identifying a microorganism in a biological sample is described where: a biological sample is obtained; DNA is extracted from the sample for subsequent PCR analysis; one or more pathogens are identified; results then undergo Application Programming Interface (API) integration using computer code with the empiric antibiotic database; and a report is generated identifying empiric antibiotic therapy.

According to the present invention as described above, since it is possible to allow effective antibiotics to be selected within a short time period, there is an advantage that it is possible to handle a large amount of biological samples in a short period of time with minimum labor and provide for an antibiotic stewardship and selection process.

Another advantage is that it is possible to automatically perform the identification of the microorganism and the antibiotic selection, thereby allowing effective antibiotics to be selected in a short period of time.

Another advantage is that antibiotic therapy can be initiated at the onset of infection alleviating the need for unnecessary and unwarranted medical care, thereby reducing healthcare costs.

It is an object of the present invention to have the decision-making process in the emergency room be influenced by the results of the assay and antimicrobial report, with the treatment and disposition of the patient altered. This, in turn, will obviate the necessity for hospitalizing some patients, providing the opportunity to institute appropriate medical therapy and reduce health care costs.

It is an object of the embodiments of the present invention to provide a PCR-based method which allows for rapid and accurate identification of pathogens in a biologic sample and informed early therapeutic intervention with oral antimicrobial agents for ambulatory patients.

It is an object of the embodiments of the present invention to link a database of antimicrobial stewardship for optimal treatment and expeditious decision-making about appropriate antibiotic therapy with the results of PCR-based pathogen assays. In some embodiments the empiric database as described herein may aid antimicrobial stewardship efforts and lead to improved patient outcomes.

It is an object of the present invention to provide clinicians with fully actionable empiric oral antibiotic recommendations from PCR-based pathogen identification.

It is an object of the present invention to provide PCR identification of the pathogen and antibiotic recommendations which correlate with clinical significance.

In one embodiment of the present invention, a method for analyzing a fluid sample for pathogens using PCR-based methods is linked via API to a database of oral antimicrobial regimens so that both physicians and patients benefit from less repetitive testing and elimination of wait times for traditional laboratory results and antibiotic sensitivity testing.

In some embodiments, API integration with the PCR technology platform allows for concurrent antimicrobial stewardship recommendations without having to exit the PCR workflow.

In some embodiments, through API integration, users will have the ability to search, navigate, and reference empiric therapy content without exiting the technology platform.

In some embodiments, the selection of the oral antibiotic therapy is based upon the pathogen identified, and clinical guidelines, published clinical trials, and/or risk factors for antimicrobial resistance contained within an empiric antimicrobial therapy database.

In some embodiments the PCR technology platform is integrated with data management systems, locally, regionally and nationally to allow for effective epidemiological surveillance with obvious benefits for antibiotic selection and control of disease outbreaks.

In some embodiments, the patient may be seen in an acute care setting such as an emergency department or hospital and the database includes intravenous and oral antimicrobial therapy.

In some embodiments, the pathogen is fungal, viral, or parasitic. Disease causing microorganisms that can be evaluated according to the method disclosed herein include different strains of bacteria, viruses, fungi, parasites, or combination thereof. However, the pathogen can be defined as any antigen that stimulates the immune system. This would include environmental antigens such as pollen.

The invention is directed to obtaining a biological sample from a patient. Sample materials may be liquid, solid or mixed liquid and solid. Preferably, the biological sample is the urine, blood, saliva, sputum, respiratory secretions including throat swabs, gastrointestinal secretions, cerebrospinal fluid, vaginal secretions, prostate secretions, wound exudates, tissue, or feces. Most preferably, the biological sample is urine, blood or sputum.

Another embodiment of the invention comprises kits for PCR sequencing and analysis according to the methods disclosed herein.

In one embodiment of the present invention, a kit is used for diagnosing or confirming a species of bacteria causing an infection in a patient with connectivity through the Cloud, an App, a computer, artificial intelligence (AI), e.g. Chat GPT or through other electronic means.

In some embodiments, nucleic acid amplification and detection methods useful for the diagnosis and management of a variety of infectious diseases other than PCR may be used.

### BRIEF DESCRIPTION OF THE DRAWINGS

FIG. 1 illustrates the schematic of PCR-based diagnostics for infectious diseases.

FIG. 2 illustrates a platform showing connections between the PCR-based test and the empiric antibiotic regimen database.

FIG. 3 is an example of a flow chart for microorganism identification by PCR with API integration for database searching.

FIG. 4 illustrates a flowchart according to example embodiments of the present disclosure.

FIG. 5 represents a comparison of culture-based antimicrobial resistance detection method and the PCR-based empiric database for urine samples of patients.

### DETAILED DESCRIPTION

The present invention overcomes disadvantages associated with current strategies and provides tools, and other methods to facilitate and simplify microorganism detection via amplification methods and provide empiric antibiotic selection. A method for molecular testing is used to identify pathogens in a biological sample. Via API integration with computer code that result is linked to a database for clinical sensitivity. The method used has a sensitivity of between 90-99%.

Aspects of the disclosure described herein may aid antimicrobial stewardship efforts and lead to improved patient outcomes. The availability of an antimicrobial therapy recommendation in a few hours or less, as opposed to a few days, could potentially decrease morbidity and mortality in ill patients due to delays in administration of appropriate therapy. The clinician can treat the patient before they even leave the emergency room or medical office. Culture-based sensitivity testing is time consuming, technologically challenging, and is often cost prohibitive, especially in resource limited countries.

Preferably, the method from testing the biological sample to selection of empiric antibiotic is performed within 4 hours or less, within 3 hours or less, or within 2 hours or less. The significantly shorter waiting period for results, can result in a tremendous decrease in healthcare costs.

The method can be used to identify pathogens in a wide variety of conditions such as urinary tract infections, sexually transmitted diseases, wounds, dermatologic infections and respiratory infections.

Preferably, the method detects genetic variations of an organism having multiple drug resistances. In some embodiments genetic loci are identified that confer resistance to agents such as rifampin, isoniazid, cephalosporins, ampicillin, fluoroquinolones, linezolid, and oseltamivir. The method detects one or more nucleic acid variations associated with drug resistance in a biological sample containing a microorganism. The method avoids such problems as false negative results from patients who previously received antibiotics.

In one aspect of the invention, other nucleic acid amplification technologies may be used to test the biological sample. Any molecular amplification method may be used to detect the pathogens. In some embodiments PCR methods are used. According to one embodiment the PCR technology may be real-time PCR (quantitative PCR or qPCR), reverse-transcriptase (RT-PCR), multiplex PCR or nested PCR.

According to one aspect of the invention, the PCR amplification process may use, but not be limited to, Thermal Cycle Machines. Specific examples of thermal cycle machine which may be used include those made by Roche Diagnostics (6800, 8800 COBAS), Vela Diagnostics (Senotas), Thermo Fischer (Quant Studio Flex 12 K).

In some embodiments, the extraction of bacterial or fungal DNA from the patient sample is performed using the MagMAX Viral/Pathogen Ultra Nucleic Acid Isolation Kit (Life Technologies). After extraction of DNA, the samples are run on the QuantStudio 12K Flex Real-Time PCR using ThermoFisher's TaqMan Array Card and Block. As an example, the TaqMan Array Urinary Tract Microbiota Specialty Comprehensive Card contains pre-plated, dried down TaqMan assays for urinary tract microbiota profiling. Results are analyzed based off of specific criteria identified during validation.

TaqMan™ PCR permits the testing of multiple biologic samples at the same time. In some embodiments the method uses commercial full panel dried antimicrobial plates such as the TaqMan assays (ThermoFisher). As an example, the urinary tract infection (UTI) TaqMan cards technology utilizes real time PCR amplification to detect presence of a microorganism in a urine sample by amplifying the genomic DNA of the organism.

Amplification and detection is performed using an Applied Biosystems™ QuantStudio™ 12K Flex real time PCR system which includes the QuantStudio™ 12 k Software. Real time PCR amplification is performed using TaqMan™ assays from Thermo Fisher Scientific consisting of two PCR primers and one fluorescently labeled (FAM dye) probe which hybridizes to the target organism's genomic DNA. The assays are preloaded onto TaqMan Array Cards (TAC).

A TaqMan™ Array Card is a 384-well microfluidic card that is prepared with dried-down TaqMan™ Assays. With an array card, gene expression is measured using the comparative Ct (ΔΔCt) method of relative quantitation. Each card can run 1 to 8 samples against 12 to 384 TaqMan™ Assay targets (including controls). The TaqMan Array Card has a small-volume design that minimizes sample and reagent consumption, and a streamlined reaction setup that saves time and reduces labor-intensive steps. It provides access to high-throughput, 384-well format without liquid-handling robotics. It has a two-fold discrimination detection at the 99.7% confidence level with standardization across multiple samples and laboratories.

In some embodiments, a sample is collected from a patient which is subjected to PCR to identify genetic markers (genes) associated with antimicrobial resistance. This PCR based detection of antimicrobial resistance genes is integrated with an empiric antibiotic database. National guidelines have reported clinical thresholds for selection of empiric therapies with not more than 10% to 20% resistance-potential for serious infections. The database encompasses a comprehensive collection of antibiotic resistance profiles, covering a wide range of bacterial strains and their associated susceptibility and resistance patterns. These profiles are built upon extensive data from NCBI, CDC, Infectious Disease Society of America (IDSA), and online antimicrobial resistance databases like ATLAS and SENTRY. When the PCR analysis detects antimicrobial resistance genes, the results are cross-referenced with the empiric antibiotic database.

This comparison allows healthcare providers to make informed decisions regarding suitable antibiotic therapies for the patient.

Certain embodiments of the present invention include, but are not limited to, the method of the invention as follows:
1) A biologic specimen is collected from the patient
2) DNA is prepared and extracted from cell (extraction process)
3) PCR amplification process (Cycle Thresholds)
4) Detection of Amplificated DNA/RNA
5) Detection of specific pathogen or pathogens
6) API integration with the Laboratory Information Management System (LIMS) system
7) Once result is processed in LIMS, API integration with empiric Database identifies the pathogen and the necessary sensitivity and resistance
8) LIMS system integrates details that correspond with the pathogen present onto the report
9) Report is generated and send to physician/patient for review

Exemplary specimens in step one are biological samples which may be whole blood (or a fraction thereof, such as plasma or serum), white blood cells, red blood cells, respiratory samples (such as bronchoalveolar lavage, sputum, saliva, mucus, oropharyngeal swab, or nasopharyngeal swab), urine, or other bodily fluids, such as tears, exudates, aspirates, punctuates, semen, vaginal fluids, amniotic fluid, spinal or cerebrospinal fluid, peritoneal effusions, plural effusions, epithelial smears, biopsies, bone marrow samples, fluids from cysts or abscesses, lavage, swabs, or any combination thereof, to perform identification of the species of bacteria present in the sample. The specimen may be human or veterinary.

In some embodiments, the specimen may be obtained from any patient (or host or subject) and any animal species, including human and non-human primates, avians, reptiles, amphibians, bovines, canines, caprines, cavities, cornvines, epines, equines, felines, hircines, lapines, leporines, lupines, ovines, porcines, racines, vulpines, including domesticated livestock, herding or migratory animals or birds, companion animals, pets, and any animal under the care of a veterinary provider.

The present disclosure will be described with respect to particular embodiments and with reference to certain drawings, but the present disclosure is not limited thereto but only by the claims. The figures described are only schematic and are non-limiting.

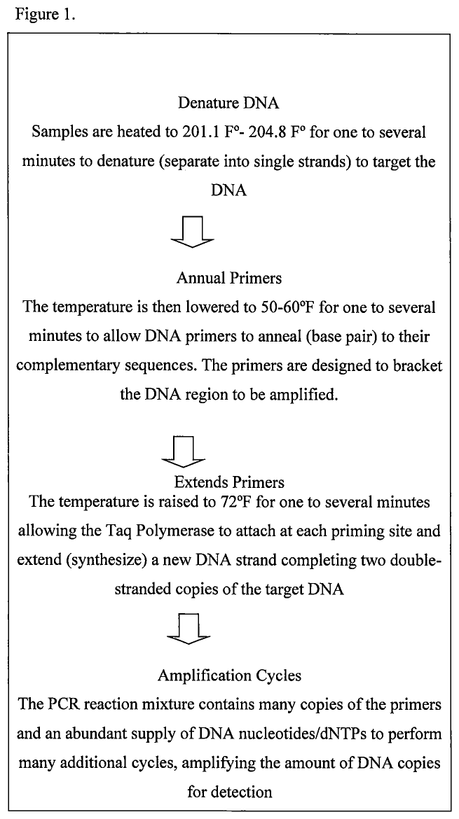

The PCR reaction takes place in a thermocycler. As shown in FIG. 1, each PCR cycle consists of three major steps: (1) denaturation of template DNA into single-stranded DNA; (2) primers annealing to their complementary target sequences; and (3) extension of primers via DNA polymerization to generate new copy of the target DNA. At the end of each cycle the newly synthesized DNA act as new targets for the next cycle. Subsequently, by repeating the cycle multiple times, logarithmic amplification of the target DNA occurs. The DNA extraction is step two (2). Amplification as in step three (3) is achieved in three (3) steps which consist of (1) denaturation, in which double-stranded DNA templates are heated to separate the strands; (2) annealing, in which short DNA molecules called primers bind to flanking regions of the target DNA; and (3) extension, in which DNA polymerase extends the 3′ end of each primer along the template strands. These steps are repeated (“cycled”) 25-35 times to exponentially produce exact copies of the target DNA. Once those cycles are completed, the amplified DNA is given a primer which illuminates certain DNA pairs in the sample. This then gives the machine a sense of how much DNA or RNA is present in the sample.

According to one aspect of the invention, a database system is provided. One or multiple databases can be searched using any known search algorithm. A database is a collection of organized information in a form which can be searched and retrieved by a computer, or other electronic processing means. As described above, the searching can be accomplished using any suitable, effective, search algorithm that can determine the presence of entries in the database.

The report, provides patient-specific details, the microorganism identified, the antibiotic such organism is sensitive to, antibiotics where there may be resistance, the dosage and regimen. Preferably, the antibiotic route is oral.

In one embodiment, the test is a PCR test which can comprise a kit for rapid Point of Care testing linked via API to a remote database for sensitivity information. POC testing is defined as testing characterized by near patient testing independent from the operation activities during the analytic procedure. In POC testing the analytical methods are performed by non-laboratory professional outside of a laboratory setting. This serves to improve patient access where lab testing is impractical, or for remote geographical areas, or areas with limited resources or for individuals who may not return for follow visits.

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example.

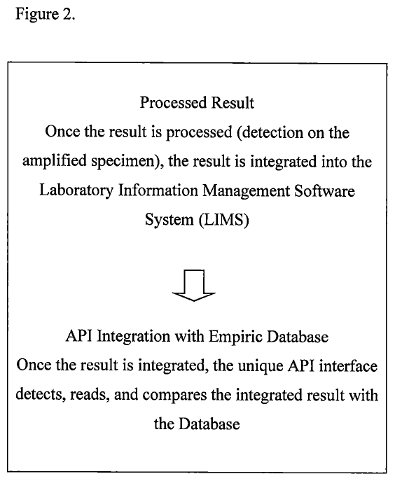

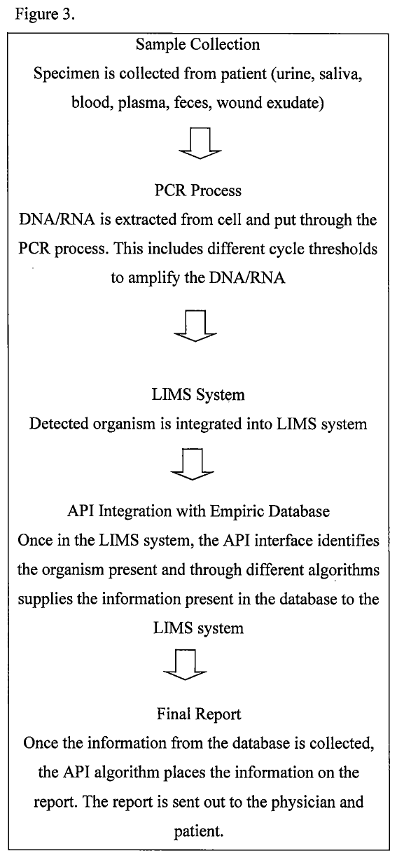

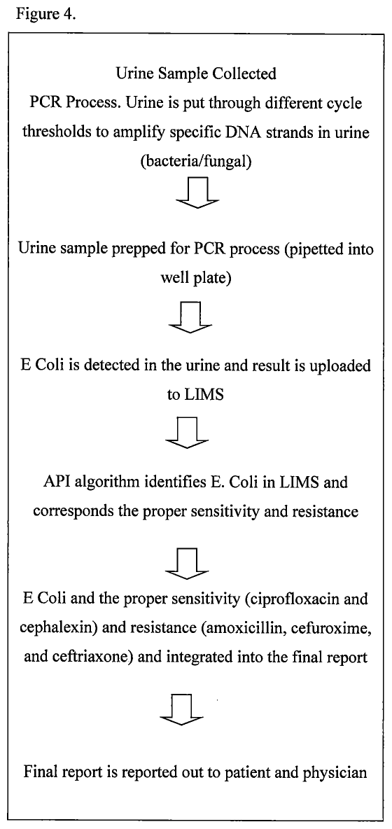

As shown in FIG. 2, a Laboratory Information Management System (LIMS) is software that allows you to effectively manage samples and associated data. By using a LIMS, the lab can automate workflows, integrate instruments, and manage samples and associated information. Via API integration the LIMS connects with the empiric antibiotic database to provide a report. As shown in FIG. 3 and FIG. 4, an API integration permits the application to exchange data with the empiric antibiotic database.

API integration is defined as the process of connecting two or more applications or systems by using Application Programming Interfaces (API) to exchange data and perform action. APIs are defined as sets of protocols and standards that allow different software applications to communicate with each other.

The terms antibiotic and antimicrobial are used interchangeably and as defined as agents used to treat or prevent bacterial, viral, fungal, parasitic, protozoan and aegaeon infections.

The term microorganism and pathogen are used interchangeably throughout and are defined as bacterial, viral, fungal, parasitic, protozoan and aegaeon.

Reference throughout this specification to “one embodiment” or “an embodiment” means that a particular feature, structure or characteristic described in connection with the embodiment is included in at least one embodiment of the present disclosure. Thus, appearances of the phrases “in some embodiments,” “in one embodiment,” or “in an embodiment” in various places throughout this specification are not necessarily all referring to the same embodiment, but may. Furthermore, the particular features, structures, or characteristics may be combined in any suitable manner, as would be apparent to one of ordinary skill in the art from this disclosure, in one or more embodiments.

Unless otherwise defined, all technical and scientific terms used herein have the same meaning as commonly understood by one of ordinary skill in the art to which this application belongs and as commonly used in the art to which this application belongs. Such art is incorporated by reference in its entirely.

Once a result is processed in LIMS, API integration with empiric Database identifies the pathogen and the necessary sensitivity and resistance. A suitable computing environment in which the API integration with the empiric database may be implemented is not limited as to scope of use or functionality, as the API integration may be implemented in diverse general-purpose or special-purpose computing systems. For example, the computing environment can be any of a variety of computing devices (e.g. desktop computer, laptop computer, server computer, tablet computer, smart phones or other mobile devices, etc.). Alternatively, the computing environment can be part of a control board. Any of the disclosed methods can be implemented as computer-executable instructions stored on one or more computer-readable storage media (e.g. one or more optical media discs, volatile memory components (such as DRAM or SRAM), or non-volatile memory components (such as flash memory or hard drives) and executed on a computer.

It should be well understood that any functionality described herein can be performed, at least in part, by one or more hardware logic components, instead of software. For example, and without limitation, illustrative types of hardware logic components that can be used include Field Programmable Gate Assays (FPGAs), Application-specific Standard Products (ASSPs), System-on-a-Chip systems (SOCs), Complex Programmable Logic Devices (CPLDs).

Any software-based embodiments can be uploaded, downloaded, or remotely accessed through suitable communication means. Such suitable communication means include, for example, the Internet, the World Wide Web, an intranet, software applications, cable (including fiberoptic cable), magnetic communications, electromagnetic communications, and other such communication means.

Where in embodiments of the present disclosure reference is made to kit for diagnosing or confirming a species of bacteria causing an infection in a patient comprising an over-the-counter device that can detect presence of such bacteria in all specimen types. Such devices can be, but not limited to, lateral flow test kits. Once the device detects the presence of specific bacteria (the device can be specifically for a certain bacteria or for multiple bacteria) the test can be scanned and paired with an App (which includes the database) that would give antibiotic sensitivity/resistance. Thus, informing the doctor via API integration (with the system) on what they could possibly prescribe or not prescribe.

Such App/API integration can also have AI algorithms to learn how to search databases to better support the present invention.

Additional factors that may affect the choice of antimicrobial regimen include the potential for inducing antimicrobial resistance, pharmacokinetic and pharmacodynamic properties, pharmacogenomics, safety profile, and cost. As such, antimicrobial stewardship guidelines are interpretative and must be based on the correlation of test results with clinical presentation and existing standards. Patients who do not demonstrate some clinical improvement within 72 hours are considered nonresponders.

In one embodiment, the method can identify heteroresistant strains. Preferably, the method identifies one of more nucleic acid variations, which may be attributed to infectious microorganisms, associated with drug resistance in a biological sample containing infectious agents.

In one embodiment, the method can identify the microorganism and the patient's pharmacogenomic profile for determination of appropriateness of a particular antibiotic selected. Preferably, the report identifies the susceptible antibiotic therapy, including but not limited to, the effective therapeutic dose.

In some embodiments, the report, i.e. test results, pathogen identified and empiric antibiotic sensitivity can be captured in the patient's electronic medical record (EMR) via API. In other embodiments, the report can be captured in local and international surveillance databases.

Disease causing organisms that can be evaluated according to the method disclosed herein include different strains of bacteria, virus, fungus, protozoa, archaeon, and parasites, or combinations thereof. Exemplary organisms that can be identified using the PCR assay, include, but are not limited to: Staphylococcus aureus, Staphylococcus lugdunensis, coagulase-negative Staphylococcus species (Staphylococcus epidermidis, Staphylococcus haemolyticus, Staphylococcus hominis, Staphylococcus capitis, not differentiated), Enterococcus faecalis, Enterococcus faecium (Enterococcus faecium and other Enterococcus spp., not differentiated, excluding Enterococcus faecalis), Streptococcus pneumoniae, Streptococcus pyogenes, Streptococcus agalactiae, Streptococcus spp., (Streptococcus mitis, Streptococcus pyogenes, Streptococcus gallolyticus, Streptococcus agalactiae, Streptococcus pneumoniae, not differentiated), Pseudomonas aeruginosa, Acinetobacter baumannii, Klebsiella spp. (Klebsiella pneumoniae, Klebsiella oxytoca, not differentiated), Escherichia coli, Enterobacter spp. (Enterobacter cloacae, Enterobacter aerogenes, not differentiated), Proteus spp. (Proteus mirabilis, Proteus vulgaris, not differentiated), Citrobacter spp. (Citrobacter freundii, Citrobacter koseri, not differentiated), Serratia marcescens, Candida albicans, and Candida glabrata.

Other specific bacteria that can be detected with the disclosed systems and methods, include without limitation: Acinetobacter baumannii, Actinobacillus spp., Actinomycetes, Actinomyces spp. (such as Actinomyces israelii and Actinomyces naeslundii), Aeromonas spp. (such as Aeromonas hydrophila, Aeromonas veronii biovar sobria (Aeromonas sobria), and Aeromonas caviae), Anaplasma phagocytophilum, Alcaligenes xylosoxidans, Actinobacillus actinomycetemcomitans, Bacillus spp. (such as Bacillus anthracis, Bacillus cereus, Bacillus subtilis, Bacillus thuringiensis, and Bacillus stearothermophilus), Bacteroides spp. (such as Bacteroides fragilis), Bartonella spp. (such as Bartonella bacilliformis and Bartonella henselae, Bifidobacterium spp., Bordetella spp. (such as Bordetella pertussis, Bordetella parapertussis, and Bordetella bronchiseptica), Borrelia spp. (such as Borrelia recurrentis, and Borrelia burgdorferi), Brucella sp. (such as Brucella abortus, Brucella canis, Brucella melintensis and Brucella suis), Burkholderia spp. (such as Burkholderia pseudomallei and Burkholderia cepacia), Campylobacter spp. (such as Campylobacter jejuni, Campylobacter coli, Campylobacter lari and Campylobacter fetus), Capnocytophaga spp., Cardiobacterium hominis, Chlamydia trachomatis, Chlamydophila pneumoniae, Chlamydophila psittaci, Citrobacter spp. Coxiella burnetii, Corynebacterium spp. (such as, Corynebacterium diphtheriae, Corynebacterium jeikeum and Corynebacterium), Clostridium spp. (such as Clostridium perfringens, Clostridium difficile, Clostridium botulinum and Clostridium tetani), Eikenella corrodens, Enterobacter spp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus spp. (such as Enterococcus faecalis and Enterococcus faecium) Ehrlichia spp. (such as Ehrlichia chafeensia and Ehrlichia canis), Erysipelothrix rhusiopathiae, Eubacterium spp., Francisella tularensis, Fusobacterium nucleatum, Gardnerella vaginalis, Gemella morbillorum, Haemophilus spp. (such as Haemophilus influenzae, Haemophilus ducreyi, Haemophilus aegyptius, Haemophilus parainfluenzae, Haemophilus haemolyticus and Haemophilus parahaemolyticus, Helicobacter spp. (such as Helicobacter pylori, Helicobacter cinaedi and Helicobacter fennelliae), Kingella kingii, Klebsiella spp. (such as Klebsiella pneumoniae, Klebsiella granulomatis and Klebsiella oxytoca), Lactobacillus spp., Listeria monocytogenes, Leptospira interrogans, Legionella pneumophila, Leptospira interrogans, Peptostreptococcus spp., Moraxella catarrhalis, Morganella spp., Mobiluncus spp., Micrococcus spp., Mycobacterium spp. (such as Mycobacterium leprae, Mycobacterium tuberculosis, Mycobacterium intracellulare, Mycobacterium avium, Mycobacterium bovis, and Mycobacterium marinum), Mycoplasm spp. (such as Mycoplasma pneumoniae, Mycoplasma hominis, and Mycoplasma genitalium), Nocardia spp. (such as Nocardia asteroides, Nocardia cyriacigeorgica and Nocardia brasiliensis), Neisseria spp. (such as Neisseria gonorrhoeae and Neisseria meningitidis), Pasteurella multocida, Plesiomonas shigelloides. Prevotella spp., Porphyromonas spp., Prevotella melaninogenica, Proteus spp. (such as Proteus vulgaris and Proteus mirabilis), Providencia spp. (such as Providencia alcalifaciens, Providencia rettgeri and Providencia stuartii), Pseudomonas aeruginosa, Propionibacterium acnes, Rhodococcus equi, Rickettsia spp. (such as Rickettsia rickettsii, Rickettsia akari and Rickettsia prowazekii, Orientia tsutsugamushi (formerly: Rickettsia tsutsugamushi) and Rickettsia typhi), Rhodococcus spp., Serratia marcescens, Stenotrophomonas maltophilia, Salmonella spp. (such as Salmonella enterica, Salmonella typhi, Salmonella paratyphi, Salmonella enteritidis, Salmonella cholerasuis and Salmonella typhimurium), Serratia spp. (such as Serratia marcesans and Serratia liquifaciens), Shigella spp. (such as Shigella dysenteriae, Shigella flexneri, Shigella boydii and Shigella sonnei), Staphylococcus spp. (such as Staphylococcus aureus, Staphylococcus epidermidis, Staphylococcus hemolyticus, Staphylococcus saprophyticus), Streptococcus spp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema spp. (such as Treponema carateum, Treponema petenue, Treponema pallidum and Treponema endemicum, Tropheryma whippelii, Ureaplasma urealyticum, Veillonella sp., Vibrio spp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia spp. (such as Yersinia enterocolitica, Yersinia pestis, and Yersinia pseudotuberculosis) and Xanthomonas maltophilia among others.

Other specific fungi that can be detected with the disclosed systems and methods, include without limitation: Candida spp. (such as Candida albicans, Candida glabrata, Candida tropicalis, Candida parapsilosis, and Candida krusei), Aspergillus spp. (such as Aspergillus fumigatous, Aspergillus flavus, Aspergillus clavatus), Cryptococcous spp. (such as Cryptococcus neoformans, Cryptococcus gattii, Cryptococcus laurentii, and Cryptococcus albidus), Fusarium spp. (such as Fusarium oxysporum, Fusarium solani, Fusarium verticillioides, and Fusarium proliferatum), Rhizopus oryzae, Penicillium marneffei, Coccidiodes immitis, and Blastomyces dermatitidis.

The various viruses that can be detected according to the methods of the present invention include, but are not limited to, adeno-associated virus, chikungunya virus, cowpox virus, coxsackie virus, dengue virus, Eastern Equine encephalitis virus, ebola virus, Epstein-barr virus, hepatitis A, hepatitis B, hepatitis C, coronavirus, cytomegalo virus, enterovirus, herpes virus, human immunodeficiency virus, papillopavirus, parainfluenza, respiratory syncytial virus, rhino virus, SARS coronavirus, influenza A, influenza B, influenza C, Japanese encephalitis virus, measles virus, monkeypox virus, mumps virus, Norwalk virus, polio virus, rabies virus, rota virus, rubella virus, SARS coronavirus 2, vaccinia virus, West Nile virus, Yellow fever virus, and zika virus.

The microorganisms (i.e. bacteria, virus, fungus, archaeon, parasite, protozoa) may be one or more strain, species, genus, class, order, family or kingdom of microorganism.

The database may contain regimens for the following antimicrobial agents mapped to the pathogen: amikacin, ampicillin, ampicillin-sulbactam, aztreonam, ceftazidime, ceftaroline, cefazolin, cefepime, ceftriaxone, ciprofloxacin, colistin, daptomycin, oxycycline, erythromycin, ertapenem, gentamicin, imipenem, linezolid, meropenem, minocycline, piperacillin-tazobactam, trimethoprim-sulfamethoxazole, tobramycin, vancomycin, or combinations of two or more thereof. Additional antimicrobial agents that may be used in the systems and methods disclosed herein also include aminoglycosides (including but not limited to kanamycin, neomycin, netilmicin, paromomycin, streptomycin, and spectinomycin), ansamycins (including but not limited to rifaximin), carbapenems (including but not limited to doripenem), cephalosporins (including but not limited to cefadroxil, cefalotin, cephalexin, cefaclor, cefprozil, fecluroxime, cefixime, cefdinir, cefditoren, cefotaxime, cefpodoxime, ceftibuten, and ceftobiprole), glycopeptides (including but not limited to teicoplanin, telavancin, dalbavancin, and oritavancin), lincosamides (including but not limited to clindamycin and lincomycin), macrolides (including but not limited to azithromycin, clarithromycin, dirithromycin, roxithromycin, telithromycin, and spiramycin), nitrofurans (including but not limited to furazolidone and nitrofurantoin), oxazolidinones (including but not limited to posizolid, radezolid, and torezolid), penicillins (including but not limited to amoxicillin, flucloxacillin, penicillin, amoxicillin/clavulanate, and ticarcillin/clavulanate), polypeptides (including but not limited to bacitracin and polymyxin B), quinolones (including but not limited to enoxacin, gatifloxacin, gemifloxacin, levofloxacin, lomefloxacin, moxifloxacin, naldixic acid, norfloxacin, trovafloxacin, grepafloxacin, sparfloxacin, and temafloxacin), suflonamides (including but not limited to mafenide, sulfacetamide, sulfadiazine, sulfadimethoxine, sulfamethizole, sulfamethoxazole, sulfasalazine, and sulfisoxazole), tetracyclines (including but not limited to demeclocycline, doxycycline, oxytetracycline, and tetracycline), and others (including but not limited to clofazimine, ethambutol, isoniazid, rifampicin, arsphenamine, chloramphenicol, fosfomycin, metronidazole, tigecycline, and trimethoprim), or any combination of two or more thereof.

Further antimicrobial agents include antifungal agents such as polyene antifungal drugs: amphotericin B, nystatin and pimaricin; azole antifungal drugs such as ketoconazole, fluconazole, itraconazole, posaconazole, voriconazole, allylamine and morpholine antifungal drugs such as naftifine and terbinafine, antimetabolite antifungal drugs such as flucytosine and other antifungal agents such as anidulafungin, caspofungin, micafungin, or any combination of two or more thereof.

Further antimicrobial agents may include antiviral drugs such as direct virus-targeting antiviral drugs include attachment inhibitors, entry inhibitors, uncoating inhibitors, protease inhibitors, polymerase inhibitors, nucleoside and nucleotide reverse transcriptase inhibitors, nonnucleoside reverse-transcriptase inhibitors, and integrase inhibitors.

Examples of protease inhibitors include darunavir, atazanavir, and ritonavir. Examples of viral DNA polymerase inhibitors include acyclovir, valacyclovir, valganciclovir, and tenofovir, and an example of an integrase inhibitor is raltegravir or any combination of two or more thereof.

This listing is not intended to be limiting. Other antimicrobial drugs known in the art may be used.

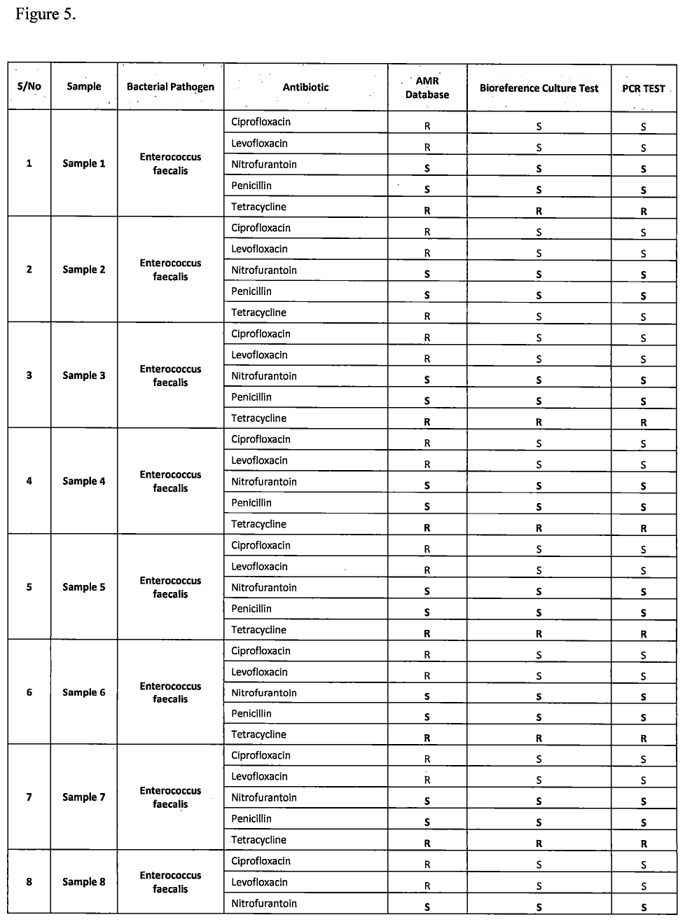

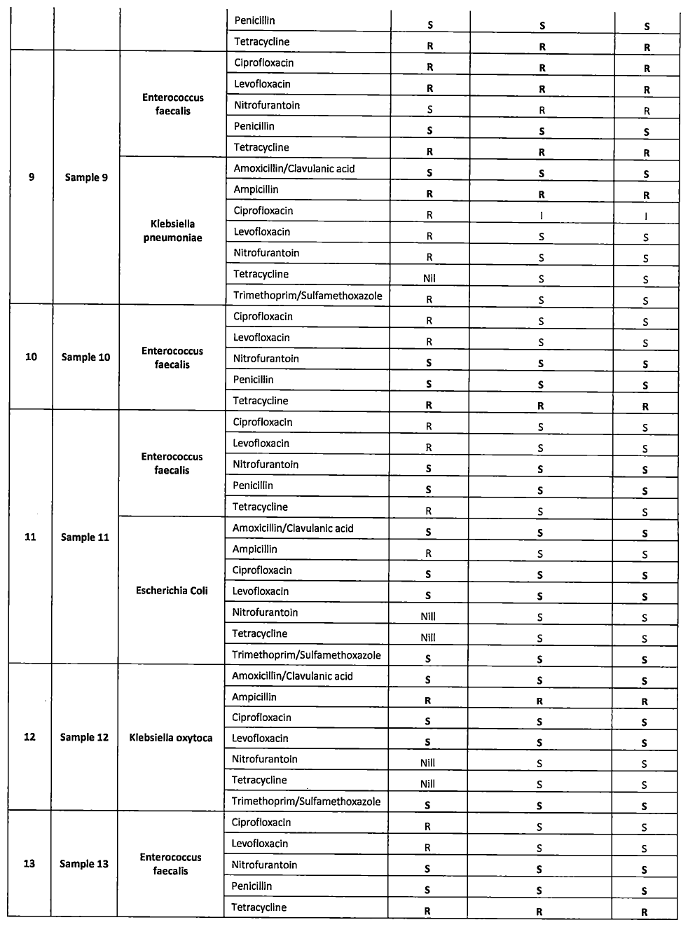

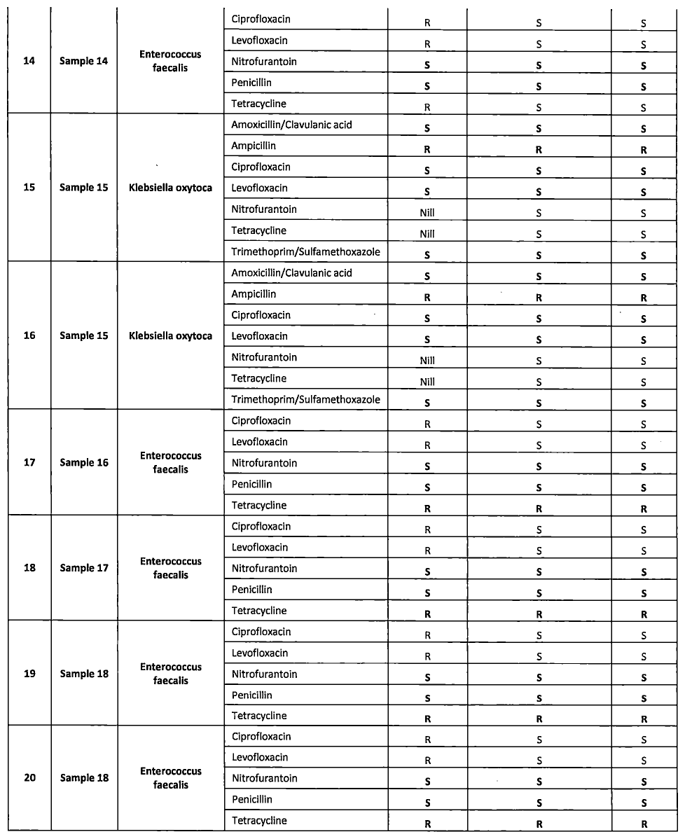

FIG. 5 shows the results of a comparison study of the antimicrobial resistance database/PCR/Culture in urine samples. For the comparison of culture-based antimicrobial resistance (AMR) detection and empiric database the urine samples of patients were collected and processed using culture-based AMR detection method. The culture test successfully revealed resistance and sensitivity patterns of UTI pathogens such a E. coli, Klebsiella oxytoca, Enterococcus faecalis and Klebsiella pneumoniae to a number of antibiotics. Subsequently, the obtained AMR pattern was then compared with the extensive sensitivity and resistance data in the empiric antibiotic database. Upon comparison it was found that the resistance and sensitivity profile of the pathogen closely matched that of the database. Based on this analysis, the healthcare professionals prescribed an empiric antibiotic regimen tailored to target the specific antimicrobial resistance exhibited by the pathogen. This concordance between the AMR pattern obtained through culture-based detection and the resistance and sensitivity data in the database highlights the clinical utility and reinforces the reliability and accuracy of the system.

### Example 1

Example of Patient Treated Using AMR Database:

Patient X came to the doctor's office exhibiting symptoms of urinary tract infections. Based on the symptoms doctor recommended a molecular urinary tract infection PCR test. The UTI test detected Klebsiella Oxytoca, Escherichia Coli and Enterococcus Faecalis. Based on these results the AMR database suggested the following regimen for the treatment of these pathogens:

| | | Sensitive | Resistant |
|---|---|---|---|
| S/No | Bacterial pathogens | UTI Database Entry | UTI Database Entry |
| [1] | Klebsiella Oxytoca | Amoxicillin/Clavulanic acid | Ampicillin |
| | | Ciprofloxacin | |
| | | Levofloxacin | |
| | | Trimethoprim/Sulfamethoxazole | |
| | | Minocycline | |
| [2] | Enterococcus Faecalis | Penicillin | Levofloxacin |
| | | Ampicillin | Ciprofloxacin |
| | | Nitrofurantoin | Minocycline |
| | | | Tetracycline |
| | | | Aminoglycosides |
| [4] | Escherichia Coli | Moxifloxacin | Levofloxacin |
| | | Amoxicillin/Clavulanic acid | Ampicillin |
| | | Nitrofurantoin | Amoxicillin |
| | | Minocycline | Trimethoprim/sulfamethoxazole |
| | | | Ciprofloxacin |

In this example, the present invention for rapid PCR-based diagnostic test was compared to a standard method requiring a lengthy incubation for 48 or more hours. The data show that the present invention is accurately able to identify the organism in the biological sample but in much less time and then provides a list of antibiotics for empiric therapy. Thus, the invention greatly reduces the time before a patient is provided an appropriate treatment regimen.

Other types of amplification tests which may be used with the present invention include, but are not limited to: reverse transcription polymerase chain reaction (RT-PCR); reverse transcription loop-mediated isothermal amplification (RT-LAMP); and isothermal amplification including: nicking endonuclease amplification reaction (NEAR), transcription mediated amplification (TMA), loop-mediated isothermal amplification (LAMP), helicase-dependent amplification (HDA), clustered regularly interspaced short palindromic repeats (CRISPR), and strand displacement amplification (SDA).

Preferred agent may change over time due to changing resistance patterns and depends on many factors, including severity of illness. Updated and detailed treatment recommendations for each pathogen are provided.

The disclosure presented herein is believed to encompass at least one distinct invention with independent utility. While the at least one invention had been disclosed in exemplary forms, the specific embodiments thereof as described and illustrated herein are not to be considered in a limiting sense, as numerous variation are possible. Equivalent changes, compositions and methods may be made within the scope of the present disclosure, achieving substantially similar results. The subject matter of the at least one invention includes all novel and on-obvious combinations and sub-combinations of the various elements, features, functions and/or properties disclosed herein and their equivalents.

1. A method for identifying a microorganism in a biological sample comprising:
obtaining a biological sample from a patient suspected of being infected with a pathogen;
extracting DNA from the sample;
performing molecular amplification analysis;
identifying one or more pathogens;
conducting API integration with an empiric antibiotic database; and
identifying empiric antibiotic therapy.

2. The method of claim 1 wherein the molecular amplification analysis on a biological sample is a PCR-based technique.

3. The method of claim 1 wherein the pathogen comprises one or more of a bacterium, a virus, a fungus, or a parasite.

4. The method of claim 1 which is performed in 6 hours or less.

5. The method of claim 1 which is performed in 2 hours or less.

6. The method of claim 1, wherein the biological sample is selected from the group consisting of blood, blood components, nasal swabs, sputum, saliva, stool, urine, throat swabs, vaginal swabs, abscess fluid, or wound swabs.

7. The method of claim 2, comprising determining a genetic variation which identifies drug resistance.

8. The method of claim 1, wherein the empiric antibiotic therapy is an oral antibiotic.

9. The method of claim 1 comprising point of care testing.

10. A method for analyzing a biological sample for pathogens comprising using a PCR-technology platform linked via API integration to a database of empiric antimicrobial regimens.

11. The method of claim 10, wherein the PCR technology platform is integrated with data management systems, locally, regionally and nationally to allow for effective epidemiological surveillance, antibiotic selection, and control of disease outbreaks.

12. The method of claim 10, wherein the API integration permits a user to search, navigate, and reference empiric oral antimicrobial regimens without exiting the PCR-technology platform.

13. A method of identifying a microorganism in a biological sample comprising:
a biologic specimen is collected from a patient;
DNA is extracted from a cell and subject to a PCR amplification process;
one or more pathogens are identified;
API integration with the Laboratory Information Management System (LIMS) system;
the identified pathogen is processed in LIMS;
API integration with an empiric antimicrobial database identifies the pathogen and the necessary sensitivity and resistance;
the LIMS system integrates details that correspond with the pathogen present onto a report; and
the report is generated and sent to a physician or a patient for review

14. The method of claim 13, having a sensitivity of between 90-99%.

15. The method of claim 13, having a turnaround time from a collection of a biological specimen to having a report generated is less than two hours.

16. The method of claim 13, further comprising a PCR method for identifying mutation loci that confer resistance to antibiotics.

17. The method of claim 13, further comprising a kit.

18. The method of claim 13, wherein the report is captured in the patient's electronic medical record via API or is captured in local and international surveillance databases.

19. The method of claim 13, comprising a kit having connectivity through the Cloud, an App, a computer, artificial intelligence (AI) or through other electronic means.

20. The method of claim 13 wherein the API integration is implemented in computer system selected from the group consisting of a desktop computer, a laptop computer, a server computer, a tablet computer, a smart phone or a mobile device.
